(19) United States
(12) Patent Application Publication
(10) Pub. No.: US 2001/0021854 A1
(43) Pub. Date: Sep. 13, 2001

(54) Uterine manipulator

(76) Inventors: Jacques Donnez, Brussels (BE); Jacques Andre, Waterloo (BE); Xavier Moreels, Gembloux (BE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/784,832

(22) Filed: Feb. 16, 2001

(30) Foreign Application Priority Data
Feb. 18, 2000 (EP) 00 870 026.2

(51) Int. Cl. A61B 17/42

(52) U.S. Cl. 606/119

(57) ABSTRACT

The present invention relates to a uterine manipulator comprising at least (i) a handle (1) which has a longitudinal axis (10), (ii) an arm (2) which also has a longitudinal axis (20) with a distal end (21) connecting it to the handle (1) and a proximal end (22), and (iii) a head (3) which has a longitudinal axis (30) and which is attached to the proximal end (22) of the arm (2), the shape and length of said arm (2) being sufficient to allow the head (3) of said arm to be introduced into the vaginal cavity, characterized in that said head (3) is provided with a sectioning device (4) arranged on a support (5), allowing the vaginal wall to be sectioned, this sectioning device (4) being arranged at a certain distance from the longitudinal axis (30) of the head (3) and being able to perform a rotating movement, preferably of 360 degrees, about said longitudinal axis (30).

### SUBJECT OF THE INVENTION

The present invention relates to a uterine manipulator for use in laparoscopy-assisted vaginal hysterectomy or in total laparoscopic hysterectomy.

### STATE OF THE ART

The function of uterine manipulators is essentially to help surgeons to visualize the peritoneal cavity and the uterus by laparoscopy, during a check-up or diagnostic examination or during a surgical operation, in particular during a hysterectomy.

Various types of uterine manipulators are known, which consist essentially of an arm fitted with a head for introduction into the uterus via the vagina.

Whether during a simple check-up or a surgical operation on the uterus, a surgeon is confronted with a certain number of problems: inherent limited visibility, anatomical identification, manipulation of the organs.

In the particular case of a surgical operation, an additional difficulty arises from the fact that the incision must be precise and must avoid damaging the surrounding tissues.

Conventionally, when the operation is performed by laparoscopy to visualize the peritoneal cavity and the uterus, a small incision is made in the abdomen to introduce a laparoscope, while the manipulator is introduced separately into the vagina. A gas, for example CO₂, is then insufflated. When an operation is involved, the surgical act is then performed using instruments introduced via small incisions made in the groin.

A certain number of manipulators have been developed to help surgeons to visualize/intervene in the uterus and to facilitate their surgical or investigative work.

Some manipulators have a first balloon which is positioned in the uterus and which, when inflated, allows the manipulator to be fixed to the wall of the uterus and allows the uterus to be moved. When these manipulators are fitted with means for introducing liquids such as contrast liquids into the uterine cavity in order to carry out a diagnosis, this first balloon also prevents the backflow of these liquids via the cervix. Optionally, a second balloon positioned in the vagina may serve as an obturator to prevent losses of gas when an incision is made in the vaginal wall.

Patent U.S. Pat. No. 5,840,077 is an example of such implementations which may be used to perform a hysterectomy by laparoscopy.

The head of a manipulable arm has a vaginal extender which is blocked at the entrance to the uterus at the level of the cervix and an end piece which enters the uterus. The vaginal extender is preferably made of plastic, but may also be made of stainless steel or of a rigid gauze composite. It may be in various forms.

The vaginal extender may be in the form of a ring or barrel whose distal part has a bevelled circular rim which serves as an anatomical landmark and incision backstop, this incision thus being simplified. Windows made in its thickness assist the visualization.

However, the vaginal extender may also be in the form of a finger or a half-cupula.

As regards the arm of the manipulator, it is articulated and can pivot about its axis, thus allowing the uterus to be positioned correctly and also the operation to be facilitated.

Nevertheless, the manipulators currently available have a certain number of drawbacks.

Usually, the components arranged on the head of such manipulators are consumable and may sometimes be relatively expensive.

In addition, the fact that an obturator in the form of a balloon or an opaque cone is used prevents good visibility of the vaginal space during the initial positioning of the manipulator.

Finally, it is difficult to section the vaginal wall by laparoscopy.

### OBJECTS OF THE INVENTION

The present invention aims to overcome these drawbacks.

The present invention aims to simplify the technique of laparoscopy-assisted vaginal hysterectomy by ensuring good visibility of the organs and a reliable anatomical identification.

The present invention aims also to provide a manipulator which would make it possible to make an incision in and cut the vaginal wall directly via the vagina, thereby increasing the safety and speed of this step and thus of the surgical operation as a whole.

The present invention aims also to provide a device for correctly obturating the uterine cavity while at the same time allowing good visualization of the cervix.

### MAIN CHARACTERISTIC ELEMENTS OF THE INVENTION

The present invention is related to a uterine manipulator comprising at least (i) a handle which has a longitudinal axis, (ii) an arm which also has a longitudinal axis with a distal end connecting it to said handle and a proximal end, and (iii) a head which has a longitudinal axis and which is attached to the proximal end of said arm, the shape and length of said arm being sufficient to allow the head of said arm to be introduced into the vaginal cavity, characterized in that said head is provided with a sectioning device arranged on a support, allowing the vaginal wall to be sectioned, this sectioning device being arranged at a certain distance from the longitudinal axis of said head and being able to perform a rotating movement, preferably of 360 degrees, about the longitudinal axis of said head.

Preferably, the longitudinal axis of the head of the device and the longitudinal axis of the arm form an angle so as to adapt the orientation of the head to the anatomy of the uterus, said angle being preferably of 25°.

Preferably, the sectioning device is a bipolar or unipolar electrode, or the like.

Preferably, the support for the sectioning device is in the form of a circular belt which moves on the head by performing a rotating movement about the longitudinal axis of the head.

Preferably, the rotating movement of the belt is controlled by control means capable of performing a rotating movement which can be transmitted to the belt via transmission means.

According to a first preferred embodiment, said control means comprise a tube which has a longitudinal axis and which is essentially located inside the handle, said handle and said tube being concentric.

According to a second preferred embodiment, the handle comprises, in its proximal part, an end piece whose longitudinal axis merges with the longitudinal axis of the arm, and said control means comprise a ring or barrel with a distal part and a proximal part, the distal part of said ring or barrel being capable of engaging with the distal part of the arm and the proximal part of said ring or barrel being capable of engaging with the end piece of the handle.

In the two preferred embodiments, said transmission means comprise a shaft with a longitudinal axis, located essentially inside the arm, said arm and said shaft being concentric, said shaft being capable of performing a rotating movement about its longitudinal axis.

According to the first preferred embodiment, said transmission means also comprise a cross-brace located at the proximal end of the arm.

Advantageously, in the two preferred embodiments, the manipulator also comprises control means for controlling the positioning of the sectioning device in two positions along the longitudinal axis of the head, a so-called down position and a so-called up position, the sectioning device being able to function (undergo rotation) only when it is in the up position (the working position).

According to the first preferred embodiment, said control means comprise a lever which moves between a down position and an up position, and a collar, said collar being solidly attached to both the arm and the shaft, and said lever bearing on said collar so as to be able to rotate.

According to the second preferred embodiment, said control means consist of said ring or barrel, said ring or barrel being able to perform a translating movement between a down position and an up position, said translating movement corresponding to a sliding movement along the longitudinal axis of the end piece of the handle.

Preferably, the manipulator according to the present invention also comprises safety means which allow the sectioning device to be blocked in its up position.

According to the first preferred embodiment, said safety means comprise a guide ring able to block the lever in the up position.

Preferably, the safety means also comprise a guide ring in order to prevent the sectioning device from rotating in the down position.

According to the second preferred embodiment, said safety means consist of a pin/groove assembly, said pin and said groove having complementary shapes, said pin and said groove being located, respectively, on the proximal part of the ring or barrel and in the inner wall of the end piece of the handle, said pin/groove assembly also preventing the sectioning device from rotating in the down position (the non-working position).

Preferably, the head of the manipulator according to the present invention is in the form of a cupula.

Preferably, said cupula is made of a transparent material or of stainless metal.

Preferably, the dimensions of said cupula are such that they allow it to act as a cervical obturator.

Preferably, the cupula is provided with teeth in order to prevent any lateral movement or sliding after it has been placed in position.

Preferably, said circular belt is also made of a transparent material or of stainless metal.

The presence of the teeth also advantageously makes torsional movement of the uterus possible.

It should be noted that, in the second preferred embodiment, the arm, the cupula and the belt are electrically isolated.

Advantageously, the manipulator comprises an electrical connector to which may be attached a cable connected to a current supply in order to carry out the hysterectomy.

The electrical current is fed from the current supply to the electrode via the inner wall of the arm up to the belt, and from the belt to the electrode either via an electrical wire or even without any wire if the belt is made of stainless metal.

The present invention is also related to a uterine manipulator consisting essentially of an arm which has a distal end and a proximal end and which is provided with a head having a longitudinal axis, the shape and length of said arm being sufficient to allow said head of said arm to be introduced into the vaginal cavity, characterized in that said head is provided with a transparent cupula.

Preferably, the dimensions of said cupula are such that they allow it to act as a cervical obturator.

Said cupula is advantageously toothed.

The present invention is also related to a method for performing a hysterectomy, characterized in that it uses the uterine manipulator according to the present invention and in that it comprises the following steps:

the various components of said manipulator are assembled, with the sectioning device placed in the non-working position or down position,

the manipulator is introduced by its head into the vagina and said manipulator is positioned correctly such that its head is placed in the vaginal cavity while at the same time obturating the cervix;

the movement of the sectioning device into its working position or up position is controlled with the control means, the sectioning device being then inside the uterus;

the sectioning device is blocked in its up position with the safety means;

electrical current is fed to the sectioning device from a current supply, and

the hysterectomy is performed by imposing a rotating movement on the sectioning device via the control means.

### BRIEF DESCRIPTION OF THE DRAWINGS

FIG. 1 is longitudinal cross section view of the manipulator according to a first preferred embodiment of the present invention.

FIG. 2 is a longitudinal cross section view of the head of the manipulator according to the invention, with the electrode and the circular belt.

FIG. 3 is a longitudinal cross section view of the cupula corresponding to the head of the manipulator according to a preferred embodiment of the present invention.

FIG. 4 is a top view of the cupula corresponding to the head of the manipulator according to a preferred embodiment of the present invention.

FIG. 5 is a longitudinal cross section view of the handle with the lever and the collar according to the first preferred embodiment of the present invention.

FIG. 6 is a top view of the handle with the lever and the collar according to the first preferred embodiment of the present invention.

FIG. 7 is a longitudinal cross section view of the handle according to the second preferred embodiment of the present invention.

FIG. 8 is a longitudinal cross section view of the manipulator according to the second preferred embodiment of the present invention, without the cupula or the handle.

FIGS. 9a and 9b are longitudinal cross section views of the manipulator assembly according to the second preferred embodiment, with the sectioning device in the down position and in the up position, respectively.

FIG. 10 represents the manipulator according to the present invention as positioned in the female genital apparatus.

### DESCRIPTION OF PREFERRED EMBODIMENTS OF THE INVENTION

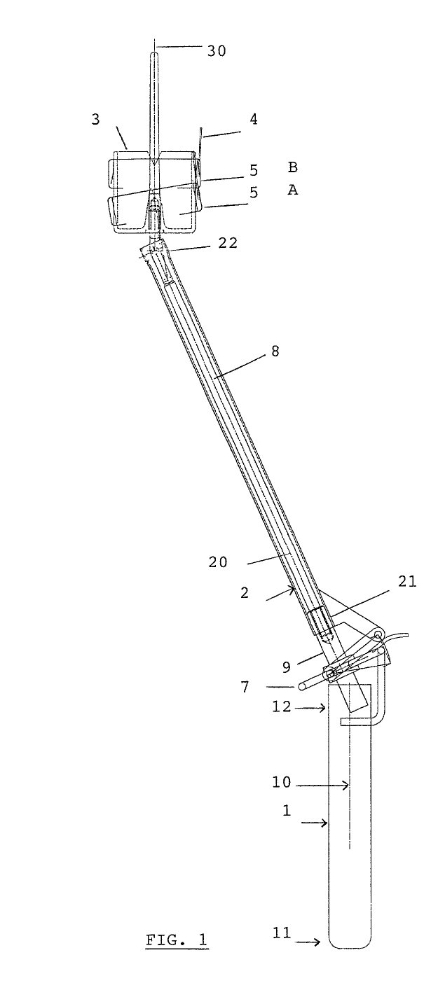

A general longitudinal cross section view of a first preferred embodiment of the manipulator according to the present invention is shown in FIG. 1.

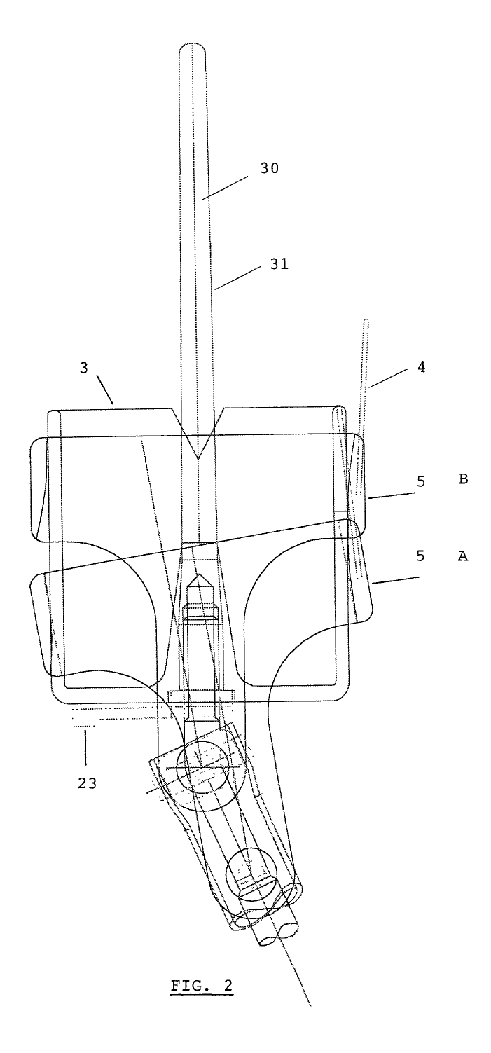

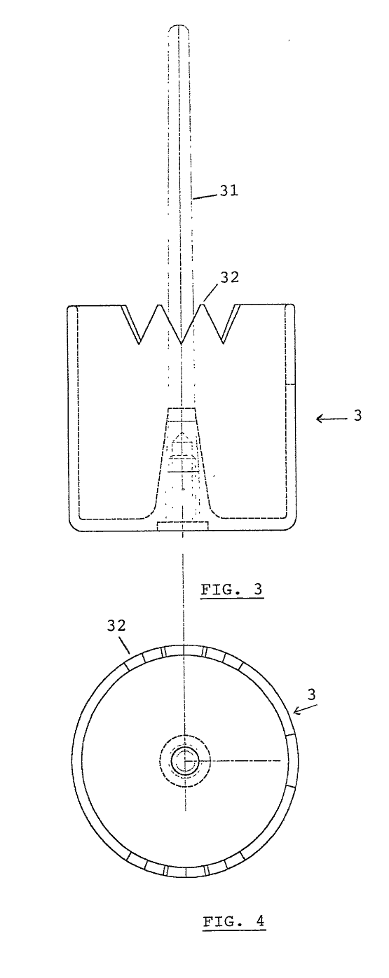

The manipulator comprises a handle 1 which has a longitudinal axis 10, a distal end 11 and a proximal end 12, an arm 2 being attached to said proximal end 12. The arm 2 itself also has a longitudinal axis 20, a distal end 21 and a proximal end 22, a head being attached to said proximal end 22. Said head is in the form of a cupula 3 with a longitudinal axis 30 and is as shown in FIGS. 2 to 4. Advantageously, the cupula 3 is made of a transparent material in order to see through it and thus to facilitate the surgical operation. The dimensions of this cupula 3 are such that it acts as a cervical obturator. The cupula 3 has at its centre an orientation device 31 capable of imposing a torsional movement on the uterus. The cupula 3 is provided with teeth 32 so as to prevent any lateral movement or sliding of the manipulator while it is being used. The teeth 32 also make, with the orientation device 31, the torsional movement of the uterus possible.

The length and shape of the arm 2 are sufficient to allow the cupula 3 to be introduced into the vaginal cavity. In addition, the longitudinal axis 20 of the arm 2 makes, with the longitudinal axis 30 of the cupula 3, a well-defined angle, preferably of 25°, so as to adapt the orientation of the cupula to the anatomy of the uterus.

Inside the arm 2 is a shaft 8, the arm 2 and the shaft 8 being concentric and the longitudinal axis of the shaft 8 thus merging with the longitudinal axis 20 of the arm 2.

A sectioning device for performing a hysterectomy is arranged inside the cupula 3. This sectioning device corresponds to an electrode 4 which may be bipolar, unipolar or the like. This electrode 4 is located at a certain distance from the longitudinal axis 30 of the cupula 3 and is able to perform a rotating movement, preferably of 360 degrees, about said longitudinal axis 30. The electrode 4 is moreover located on a support corresponding to a circular belt 5 able to move relative to the cupula 3 by rotation about the longitudinal axis 30 of the cupula 3.

Means for controlling the rotating movement of the circular belt 5 and thus that of the electrode 4 are located on the manipulator. These control means also constitute means for controlling the positioning of the circular belt 5 and thus of the electrode 4 along the longitudinal axis 30 of the cupula 3 in two positions, a down position A and an up position B.

A first control means consists of a tube (not shown) located in the handle 1 and connected to a connector 9.

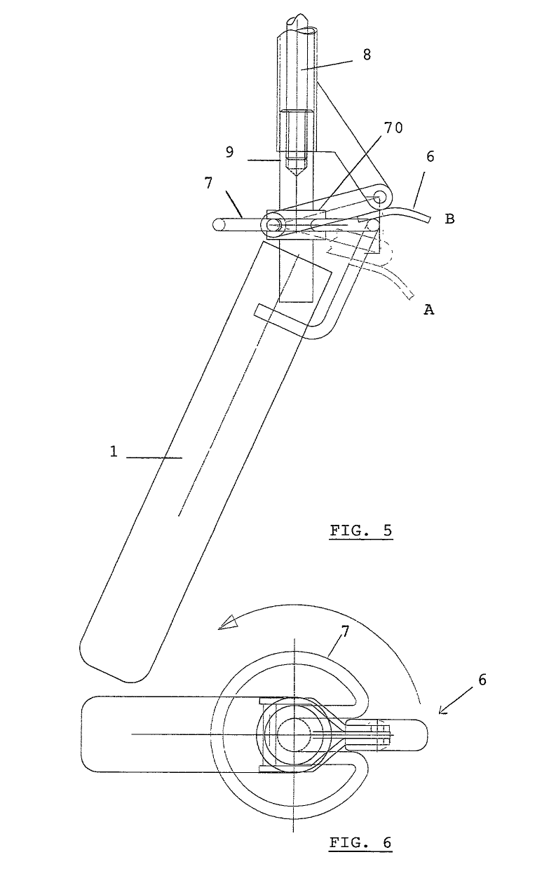

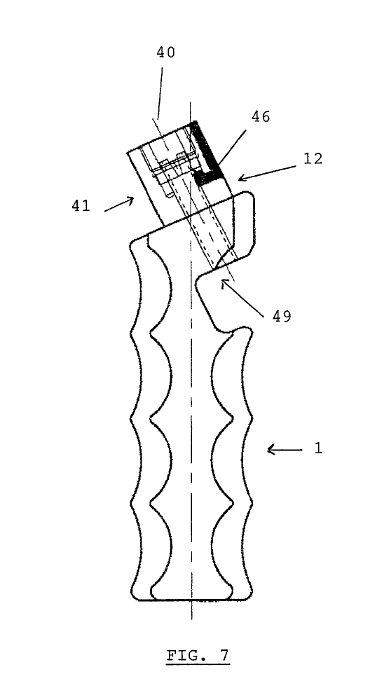

A second means for controlling the position of the circular belt 5 and thus of the electrode 4 in an up position B or down position A consists of a lever 6 capable of bearing on a collar 7, as shown in FIG. 5, and being solidly attached to both the arm 2 and the shaft 8 located inside this arm 2. The lever may be placed in two positions, a down position A and an up position B. The configuration of the collar 7 and of the lever 6 is such that the lever 6 bears on the collar 7 throughout its circular path in the up position B, but not in the down position A, as shown in FIG. 6.

Means for transmission of the rotating movement and also of the up position A or down position B of the circular belt 5 and thus of the electrode 4, from the control means to said circular belt 5 and the electrode 4, are also provided on the manipulator.

These transmission means comprise the shaft 8 of the arm 2 which is able to rotate about the longitudinal axis 20 of the arm 2 and of moving along said axis 20.

These transmission means also comprise a cross-brace 23 located at the proximal end 22 of the arm 2.

Safety means allow the manipulator to be made safe. These safety means correspond here to a guide ring 70 with a notch into which the lever 6 engages in the up position B, thus allowing it to be blocked in this position and preventing the circular belt 5 and thus the electrode 4 from rotating.

In the non-working position, that is to say when the electrode 4 is not in use, the manipulator is in a configuration in which the electrode 4 and the circular belt 5, along with the lever 6, are in the down position. In this position, the electrode 4 and the circular belt 5 are located inside the cupula 3.

The following steps should be followed to use the electrode 4:

in a first stage, the shaft located in the handle 1 is pushed, the effect of which is to actuate the lever 6 and to switch it from its down position A to its up position B. In its up position B, the lever 6 then engages with the guide ring 70, and thus blocks it in this position. The lever 6 then bears on the collar 7. The movement of the lever 6 from its down position A to its up position B has the effect, firstly, of moving the shaft 8 located inside the arm 2, and then of moving the circular belt 5 and the electrode 4 from the down position A to the up position B. In this new position, the electrode 4 projects beyond the cupula 3 and is thus ready to undergo a rotating movement;

in a second stage, the lever 6 is subjected to a rotating movement which is transmitted to the circular belt 5 and thus to the electrode 4 which can then be used for the hysterectomy.

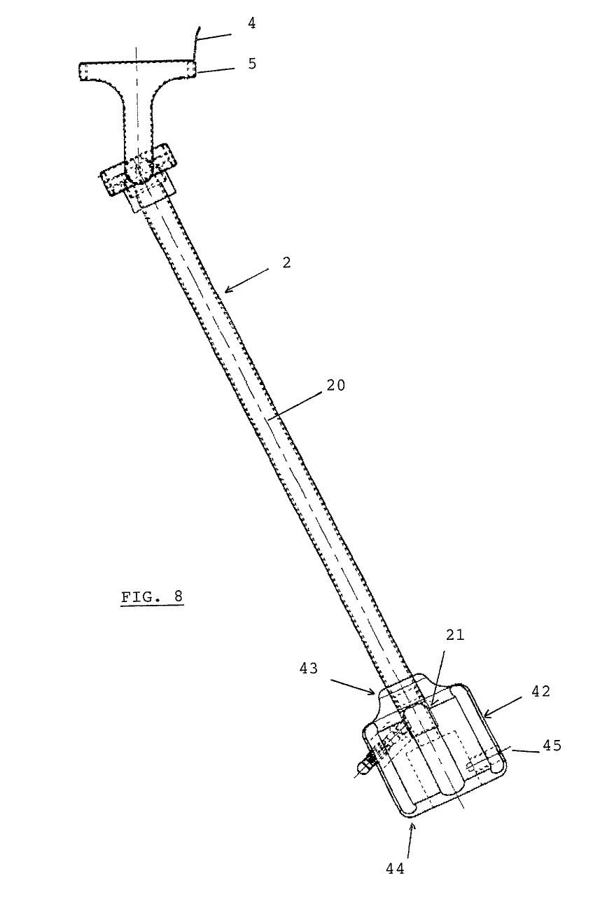

In a second preferred embodiment, the manipulator is as shown in FIGS. 7, 8, 9a and 9b. This manipulator essentially comprises, like the manipulator according to the first embodiment, a handle 1, an arm 2 and a head 3. Here also, the head is preferably in the form of a cupula 3 and is provided with a sectioning device 4, in this case an electrode, located on a belt 5. As in the first embodiment, the circular belt 5 and the sectioning device 4 are able to perform a rotating movement, preferably of 360 degrees about the longitudinal axis 30 of the head 3.

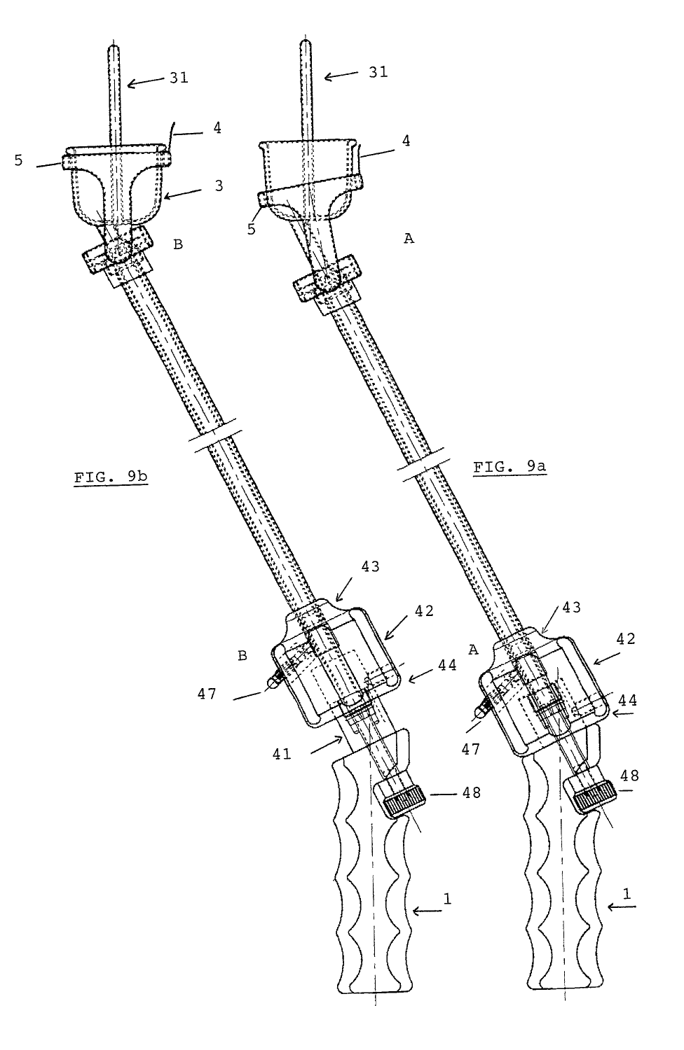

In this case also, the manipulator has a configuration such that the electrode 4 can undergo rotation only when it is in the working position, this position corresponding to an up position B as illustrated in FIG. 9b. In this position, the electrode projects beyond the cupula 3. For the rest of the time, the electrode 4 is in the so-called non-working position or down position A, as illustrated in FIG. 9a. In this position, the electrode 4 is located in the cupula 3.

The up position B or down position A is controlled by control means and is made safe by safety means, as in the manipulator according to the first embodiment. However, these means take a different form in this case.

As in the manipulator according to the first embodiment, the manipulator also comprises control means which control the rotating movement of the electrode 4. In this case also, these control means are able to perform a rotating movement which may be transmitted to the belt 5 supporting the electrode 4 via transmission means. However, these means take a different form in this case.

More precisely, the handle 1 of this manipulator, which is ergonomically shaped, is provided in its proximal part 12 with an end piece 41, as shown in FIG. 7. This end piece 41 has a longitudinal axis 40 which merges with the longitudinal axis 20 of the arm 2. Furthermore, the inside of the end piece 41 comprises a groove 46, the function of which will be described later.

The manipulator according to this second embodiment further comprises a ring or barrel 42 with a distal part 43 and a proximal part 44, as illustrated in FIG. 8. In order to use the manipulator, this ring or barrel 42 must be fixed, firstly, via its distal part 43 to the distal part 21 of the arm 2 and, secondly, via its proximal part 44 to the end piece 41 of the handle 1, as illustrated in FIGS. 8, 9a and 9b.

Once fixed to the arm 2 and to the end piece 41, the ring or barrel 42 constitutes in this embodiment the control means via which is controlled the rotating movement of the sectioning device, that is to say the belt 5 and the electrode 4. Specifically, the ring or barrel 42 is able to perform a rotating movement about the longitudinal axis 20 of the arm 2. This rotating movement may be transmitted to the belt 5 via a shaft 8 located inside the arm 2, as in the first embodiment. The shaft 8 thus acts as a transmission means.

Nevertheless, as in the first embodiment, it is envisaged that the ring or barrel 42 can control the rotation of the sectioning device only under certain conditions.

More specifically, the ring or barrel 42 is able to move between a down position A and an up position B by translation on the end piece 41 and along the longitudinal axis 20 of the arm 2. The movement of the ring or barrel 42 between the down position A and the up position B brings about the movement of the belt 5 and thus of the electrode 4 between its down position A and its up position B as defined above.

When the ring or barrel 42 is in the down position A, it cannot rotate about the axis 20 since it is blocked by safety means. These safety means consist of a pin 45/groove 46 assembly, the pin 45 being located on the ring or barrel 42, while the groove 46 is located on the end piece 41 of the handle 1. The pin 45 and the groove 46 have complementary shapes, such that the pin 45 and the groove 46 can engage with each other to block the ring or barrel 42 in the down position. In addition, this complementarity is such that when the ring or barrel 42 is switched to the up position B, it can also be blocked in this position and cannot slide and return spontaneously to the down position A. In the present case, the groove 46 is in the shape of an inverted L.

The advantage of the manipulator according to this second preferred embodiment over that according to the first embodiment is that this manipulator can be dismantled by virtue, essentially, of the presence of a cavity 49 in the handle 1, which is intended to receive a screw 48. Consequently, the manipulator also has the advantage of being easy to sterilize.

The belt 5 and the cupula 3 are preferably made of plastic or of stainless metal. Advantageously, the cupula 3 is made of transparent plastic so as to facilitate the surgeon's work by giving him or her better visibility.

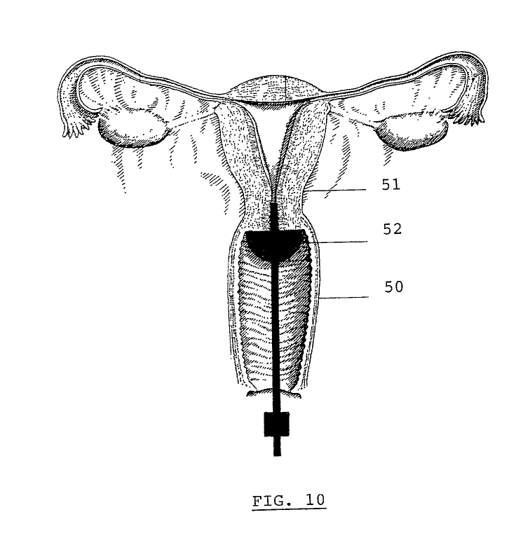

In concrete terms, when a hysterectomy is to be performed, the various components of the manipulator are assembled in a first stage, with the ring or barrel 42 and the sectioning device placed in the non-working position or down position. The shaft 8 is blocked inside the arm 2 by means of the screw 48. Next, the manipulator is introduced by its head 3 into the patient's vagina and is positioned correctly, as illustrated on FIG. 10, so that its arm 2 is located in the vagina 50 and its head 3 is blocked at the entrance to the uterus 51 and serves as an obturator at the cervix 52. Once the uterine manipulator is correctly positioned, the movement of the sectioning device 4 into the up position is controlled by moving the ring or barrel 42 into the up position B. The sectioning device is then blocked in the up position and the hysterectomy can then begin.

To do this, a current supply must be used and the electrical current must be fed to the belt 5 and the electrode 4. To this end, an electrical cable, which is from one end connected to the current supply, is connected from the other end on an electrical connector 47 located in the manipulator, preferably on the ring or barrel 42. The manipulator is such that, with the ring 2, the belt 5 and the electrode 4 electrically isolated, the current is fed to the belt 5 and the electrode 4 via the inner walls of the arm 2. If the belt 5 is made of metal, the current is conveyed from the belt 5 to the electrode 4 by the metal itself. When the belt 5 is made of plastic, the transmission takes place by means of an electrical wire.

During the operation, in order to facilitate the surgical act, one can use the orientation device 31 and the teeth 32 of the cupula 3, as disclosed in the first embodiment, for applying a torsional movement to the uterus 51.

What is claimed is:

1. A uterine manipulator, comprising:
a handle having a longitudinal axis;
an arm having a longitudinal axis, wherein said arm has a distal end and a proximal end, said distal end being connected to said handle;
a head having a longitudinal axis, wherein said head is attached to the proximal end of said arm, said head having a configuration adapted for introduction into a vaginal cavity; and
a sectioning device rotationally engaged with said head such that said sectioning device is able to rotate about said longitudinal axis of said head.

2. The uterine manipulator of claim 1, wherein the longitudinal axis of said head and the longitudinal axis of said arm form an angle which adapts the orientation of the head to the anatomy of the uterus.

3. The uterine manipulator of claim 2, wherein said angle is 25°.

4. The uterine manipulator of claim 1, wherein said sectioning device is an electrode.

5. The uterine manipulator of claim 4, wherein said electrode is a bipolar electrode.

6. The uterine manipulator of claim 4, wherein said electrode is a monopolar electrode.

7. The uterine manipulator of claim 1, wherein said sectioning device is supported by a circular belt capable of rotating about the longitudinal axis of said head.

8. The uterine manipulator of claim 7, wherein said belt is made of a material selected from the group consisting of transparent materials and stainless metals.

9. The uterine manipulator of claim 7, further comprising control components, wherein said control components control the rotational movement of said belt.

10. The uterine manipulator of claim 9, further comprising transmission components, wherein said transmission components transmit said rotational movement from said control components to said belt.

11. The uterine manipulator of claim 10, wherein said control components comprise a tube having a longitudinal axis, said tube being located essentially inside said handle such that said tube and said handle are concentric.

12. The uterine manipulator of claim 10, wherein said handle comprises an end piece whose longitudinal axis merges with the longitudinal axis of the arm, said control components comprise a ring or barrel with a distal part and a proximal part, wherein the distal part of said ring or barrel is capable of engaging the distal part of the arm and the proximal part of said ring or barrel is capable of engaging said end piece of said handle.

13. The uterine manipulator of claim 10, wherein said transmission components comprise a shaft inside the arm, said shaft having a longitudinal axis and being located essentially inside said arm, wherein said shaft and said arm are concentric and wherein said shaft is capable of rotating about its longitudinal axis.

14. The uterine manipulator of claim 13, wherein said transmission components further comprise a cross-brace at the proximal end of said arm.

15. The uterine manipulator of claim 1, further comprising control components for controlling the positioning of said sectioning device between a sectioning position and a non-sectioning position along the longitudinal axis of the head, said sectioning device being capable of rotating only when said control components are in the sectioning position.

16. The uterine manipulator of claim 15, wherein said control components comprise
a lever, wherein said lever moves between a sectioning position and a non-sectioning position; and
a collar, said collar being solidly attached to both the arm and the shaft, wherein said lever bears on said collar so as to be able to rotate.

17. The uterine manipulator of claim 15, wherein said control components comprise a ring or barrel, said ring or barrel being capable of performing a translational movement between a non-sectioning position and a sectioning position, said translational movement being a sliding movement along the longitudinal axis of the end piece of the handle.

18. The uterine manipulator of claim 15, further comprising safety components, wherein said safety components allow said sectioning device to be blocked in its sectioning position.

19. The uterine manipulator of claim 16, wherein said safety components comprise a guide ring capable of blocking said lever in said sectioning position.

20. The uterine manipulator of claim 19 wherein said guide ring prevents said sectioning device from rotating in said non-sectioning position.

21. The uterine manipulator of claim 18, wherein said safety components comprise a pin and groove assembly, said pin and said groove having complementary shapes, said pin being located on the proximal part of the ring or barrel, said groove being located in the inner wall of said end piece of said handle, wherein said assembly prevents said sectioning device from rotating in said non-sectioning position.

22. The uterine manipulator of claim 1, wherein said head comprises a cupula.

23. The uterine manipulator of claim 22, wherein said cupula is made of a material selected from the group consisting of transparent materials and stainless metals.

24. The uterine manipulator of claim 22, wherein said cupula serves as a cervical obturator.

25. The uterine manipulator of claim 22, wherein said cupula comprises teeth.

26. A method for performing a hysterectomy on a patient, comprising:
obtaining a manipulator according to claim 1;
introducing said manipulator into the patient such that said arm is located in the vagina and said head is located at the entrance to the uterus and serves as an obturator at the cervix; and
rotating said sectioning device about said longitudinal axis of said head.

27. The method of claim 26, wherein said sectioning device is introduced into the patient in a non-sectioning configuration and placed in a sectioning configuration when it is desired to perform said hysterectomy.

28. The method of claim 27, wherein said rotating step comprises rotating control components.

29. The method of claim 28, further comprising using safety components to maintain said sectioning device in said sectioning configuration.

30. The method of claim 29, further comprising applying electric current to said sectioning device.
